(19) United States
(12) Patent Application Publication
(10) Pub. No.: US 2005/0154447 A1
(43) Pub. Date: Jul. 14, 2005

(54) Ostium stent system

(75) Inventor: Justin Goshgarian, Santa Rosa, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc.

(21) Appl. No.: 10/753,461

(22) Filed: Jan. 9, 2004

(51) Int. Cl. A61F 2/06

(52) U.S. Cl. 623/1.15

(57) ABSTRACT

An ostium stent system includes a stent having a tubular body at the distal portion of the stent and a flaring portion at the proximal portion of the stent. A restraining structure that deploys the flaring portion covers only the proximal portion of the stent, providing a low profile for smooth delivery of the stent to the treatment site. The flaring portion comprises at least one flaring member which each comprise a short segment and a long segment. In an unexpanded configuration, each short and long segment are parallel to a longitudinal axis of the tubular body. When the flaring portion is deployed, each short segment remains parallel while each long segment becomes perpendicular to the longitudinal axis of the tubular body. The short segments therefore absorb the radial force resulting from deployment of the long segments and thereby prevent the stent from lifting off the balloon.

### BACKGROUND OF THE INVENTION

1. Field of the Invention

The field of this invention generally relates to a medical device, and more particularly to an expandable stent positioned at an ostium for treatment of stenosis.

2. Background Art

A wide range of medical treatments have been previously developed using “endoluminal prostheses,” which is herein intended to mean medical devices which are adapted for temporary or permanent implantation within a body lumen. Examples of lumens in which endoluminal prostheses may be implanted include, without limitation: arteries, veins, gastrointestinal tract, biliary tract, urethra, trachea, hepatic shunts, and fallopian tubes. Various types of endoluminal prostheses have been developed, each providing a uniquely beneficial structure to modify the mechanics of the targeted luminal wall.

Various stent designs are known in the art for providing radial support to wall tissue, which form the various lumens within the body. For example, stents have been designed for use in the treatment of cardiovascular disease. There are a number of methods and devices for treating coronary heart disease, some of which are specifically designed to treat the complications resulting from atherosclerosis and other forms of coronary arterial narrowing. For instance, angioplasty procedures serve to enlarge the lumen of the affected coronary artery by radial hydraulic expansion. The procedure may be accomplished by inflating a balloon of a balloon catheter within the narrowed lumen of the coronary artery. In some instances the vessel restenoses chronically, or closes down acutely, negating the positive effects of the angioplasty procedure.

To provide radial support to the treated vessel in order to prolong the positive effects of the angioplasty procedure, a stent may be implanted in conjunction with inflating the balloon. Effectively, the stent overcomes the natural tendency of the vessel walls of some patients to close back down, thereby maintaining a more normal blood flow through that vessel than would be possible if the stent were not in place. Under this procedure, the stent may be delivered to the treatment site within the affected lumen and expanded to its desired diameter for treatment.

Stents are delivered to the treatment site by a catheter device. Typically, the stent is introduced into the patient in an unexpanded form, having the smallest diameter possible. The small diameter is necessary during insertion in order to properly traverse blood vessels. When the stent reaches the treatment site, the stent is expanded to engage the blood vessel walls, enlarging the inner circumference of the blood vessel, and securing to the vessel wall.

The stent may be balloon-expandable or self-expandable. If the stent is balloon-expandable, the stent is mounted on a balloon of a balloon catheter and delivered to the treatment site. The balloon is inflated while inside the circumference of the stent, forcing the stent to expand and lodge within the blood vessel at the treatment site. If the stent is self-expandable, the stent is loaded in a retaining sheath and constructed from any appropriate material such that the stent expands upon removal of the retaining sheath.

Positioning a stent at an ostium is particularly challenging since the stent must be accurately positioned at the treatment site. At an ostium, the stent may be inserted too far or not far enough. If the stent is not correctly positioned at the ostium, the stent may either miss a portion of the treatment site and/or potentially restrict blood flow if the stent projects into the aorta. Accordingly, stents have been developed specifically for use at an ostium. An example of a stent for use at an ostium can be found in U.S. Pat. No. 5,607,444 to Lam, the disclosure of which is incorporated herein by reference. U.S. Pat. No. 5,607,444 teaches an ostial stent with an expandable tubular body and an end portion that is capable of being flared. The ostial stent is placed within the vessel with its flaring portion, flared open, at the ostium and the tubular body of the ostial stent extending over the treatment site.

One problem associated with stents designed for use at an ostium is that when the flaring portion is deployed, the deployment causes the body of the stent to raise off the balloon. Subsequently, expansion of the tubular body of the stent may be effected since the stent was slightly separated from the balloon. Another concern with all stents is that the tubular body of the stent must have adequate radial strength in order to keep the vessel open. Stents may be constructed all or in part of an elastic material that allows the stent to be self-expanding. However, elastic materials may not provide adequate radial strength to keep the treatment site open and maintain the desired blood flow for the patient.

In addition, as previously mentioned, stents are typically introduced into the patient in an unexpanded form such that the stent has the smallest diameter possible. The smallest diameter possible gives the stent a low profile, which allows the stent to properly traverse blood vessels and therefore makes delivery to the treatment site as smooth as possible. The delivery method of the stent must therefore comprise a device that maintains a low profile in order to ensure smooth delivery to the treatment site.

It is an objective of the present invention to provide for a stent for the treatment of stenoses at an ostium of blood vessels or tubular organs.

Further, it is an objective of the present invention to provide a stent to be used at an ostium that does not become separated from the balloon when the flaring portion is deployed.

It is another objective of the present invention to provide a stent for use at an ostium that comprises a tubular body of sufficient radial strength while simultaneously having a flaring portion that is flexible and self-expanding.

It is also an objective of the present invention to provide a stent for use at an ostium that comprises a device for delivery such that the stent maintains a low profile for smooth delivery to the treatment site.

### BRIEF SUMMARY OF THE INVENTION

To achieve the foregoing and other objects, and in accordance with the purposes of the present invention as embodied and broadly described herein, the ostium stent system of the present invention comprises a stent having a tubular body at the distal portion of the stent and a flaring portion at the proximal portion of the stent. The ostium stent system aids in correctly positioning the stent in an ostium.

In its unexpanded configuration, the tubular body is generally hollow and cylindrical in shape and includes a proximal end, a distal end, and a longitudinal axis. The tubular body comprises a plurality of circumferential rings that are in the shape, for example, of a sinusoid. Each circumferential ring runs continuously around the circumference of the tubular body and provides radial strength for the stent. A balloon on a balloon catheter serves to expand the tubular body, therefore allowing the stent to engage the branch vessel walls for treatment.

The flaring portion is attached to the proximal end of the tubular body and is comprised of a plurality of flaring members. The flaring members each comprise a short segment and a long segment. Each short segment is attached to the tubular body such that the short segment is parallel to the longitudinal axis of the tubular body. Each long segment is attached to the corresponding short segment and is also parallel to the longitudinal axis of the tubular body in an unexpanded configuration. However, when the flaring portion is deployed, each short segment remains parallel to the longitudinal axis of the tubular body while each long segment becomes perpendicular to the longitudinal axis of the tubular body.

The advantage of having the short segments of the flaring members remain parallel to the longitudinal axis of the tubular body after deployment of the flaring portion is that the short segments absorb the radial force resulting from deployment of the long segments and thereby prevent the stent from lifting off the balloon. Further, having the flaring members hinge as part of the flaring portion rather than at the flaring portion/tubular body junction reduces stress at the flaring portion/tubular body junction. As this junction may be welded, increased stress at the junction may cause cracking in the weld. In their expanded configuration, the long segments serve to engage the wall of a great vessel at an ostium, thereby aiding in correctly positioning the ostium stent system.

While the tubular body is balloon expandable, the flaring portion is self expandable. A restraining structure covers only the flaring portion, therefore covering only the proximal portion of the stent. When the restraining structure is retracted or removed, the flaring portion is deployed and the long segments engage the branch vessel walls at the treatment site. The advantage of having the restraining structure cover only the proximal portion of the stent as opposed to covering the entire stent is that the overall device has a lower profile. The lower profile allows the device to properly traverse blood vessels, providing smooth delivery to the treatment site.

### BRIEF DESCRIPTION OF THE FIGURES.

The foregoing and other features and advantages of the invention will be apparent from the following, more particular description of a preferred embodiment of the invention, as illustrated in the accompanying drawings.

FIG. 1 is a side view of the ostium stent system of the present invention with the flaring portion in an expanded configuration and the tubular body in an unexpanded configuration.

FIG. 1A is a side view of the ostium stent system of the present invention showing an embodiment with connecting elements connecting the rings of the tubular body.

FIG. 2 is a front view of FIG. 1, illustrating the ostium stent system of the present invention with the flaring portion in an expanded configuration and the tubular body in an unexpanded configuration.

FIG. 3 is a side view of the ostium stent system of the present invention on a balloon of a balloon catheter with both the flaring portion and the tubular body in an unexpanded configuration.

FIG. 4 shows a guide catheter being inserted into a main vessel.

FIG. 5 shows the ostium stent system of the present invention being advanced to the treatment site.

FIG. 6 shows the guide catheter being pulled back and the ostium stent system being placed proximal to the ostium.

FIG. 7 shows the flaring portion of the ostium stent system in an expanded configuration.

FIG. 8 shows the ostium stent system being advanced until the expanded flaring portion opposes the wall of the main vessel.

FIG. 9 shows the ostium stent system in position at the ostium, with both the flaring portion and the tubular body in an expanded configuration.

### DETAILED DESCRIPTION OF THE INVENTION

A preferred embodiment of the present invention is now described with reference to the figures, where like reference numbers indicate identical or functionally similar elements. Also in the figures, the left most digit of each reference number corresponds to the figure in which the reference number is first used. While specific configurations and arrangements are discussed, it should be understood that this is done for illustrative purposes only. A person skilled in the relevant art will recognize that other configurations and arrangements can be used without departing from the spirit and scope of the invention.

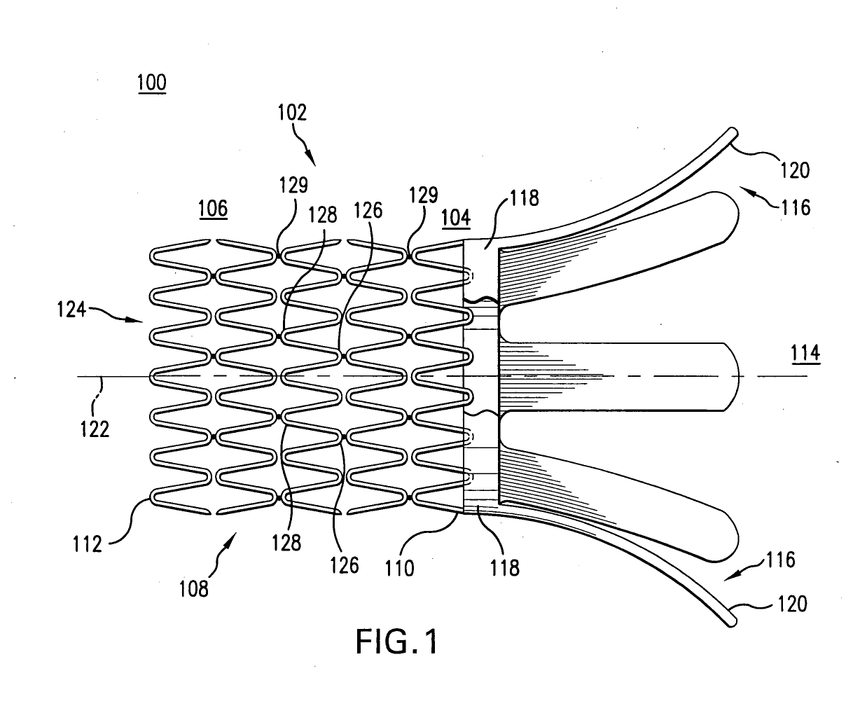

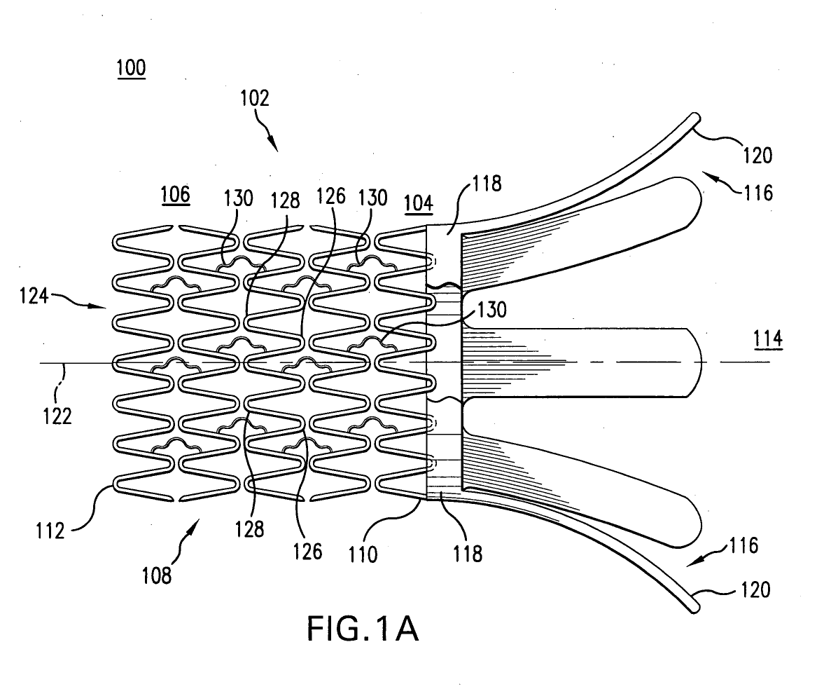

Referring to FIG. 1, an embodiment of an ostium stent system 100 of the present invention is shown. Ostium stent system 100 comprises a stent. 102, having a tubular body 108 at a distal portion 106 of stent 102 and a flaring portion 114 at a proximal portion 104 of stent 102. FIG. 1 shows a side view of ostium stent system 100 with flaring portion 114 in an expanded configuration and tubular body 108 in an unexpanded configuration. Ostium stent system 100 aids in positioning stent 102 in an ostium, as will be further described below.

In its unexpanded configuration, tubular body 108 is generally hollow and cylindrical in shape. Tubular body 108 includes a proximal end 110, a distal end 112, and a longitudinal axis 122. Tubular body 108 comprises a plurality of circumferential rings 124 that are longitudinally spaced apart along longitudinal axis 122. The circumferential rings 124 are in the shape, for example, of a sinusoid. Each circumferential ring 124 comprises a plurality of peaks 126 and valleys 128.

For purposes of illustration, the proximal end of the sinusoid has been arbitrarily labeled peak and the distal end of the sinusoid has been arbitrarily labeled valley. It would be understood by one of ordinary skill in the art that peaks 126 and valleys 128 have been labeled for illustrative purposes and that the terms may be switched without departing from the scope of the invention. Peaks 126 and valleys 128 may face either longitudinal direction provided that all peaks 126 face one longitudinal direction and all valleys face the opposite longitudinal direction. Thus, flipping circumferential rings 124 would by definition convert all the peaks to valleys and valleys to peaks.

Each circumferential ring 124 runs continuously around the circumference of tubular body 108 and provides radial strength for stent 102. FIG. 1 shows circumferential rings 124 in which peaks 126 and valleys 128 are out of phase, meaning that peaks 126 are facing valleys 128. Circumferential rings 124 are interconnected in that peaks 126 of one ring are connected to valleys 128 of a second ring. Circumferential rings 124 may be interconnected by any number of a number of different methods known in the art, such as direct welding or via a connecting element. Circumferential rings 124 also may be connected together starting with a tubular element and etching away the unwanted material, leaving circumferential rings 124 connected together at peak/valley junctions. FIG. 1 shows the circumferential rings 124 welded together at weld points 129. It is preferable that the circumferential rings are not connected together at each peak and valley for improved flexibility. As shown in FIG. 1, the weld points 129 are spaced with an unconnected peak/valley between the weld points. FIG. 1 also shows that the weld points are staggered in adjacent circumferential rings, also to improve flexibility. FIG. 1A is identical to FIG. 1 except that connecting elements 130 are utilized to connect adjacent circumferential rings 124 to each other. Connecting elements 130 can be any shape. In addition, one skilled in the art can appreciate that the circumferential rings 124 can be aligned such that peaks 126 and valleys 128 are in phase, meaning that peaks 126 of circumferential rings 124 would face each other and valleys 128 of circumferential rings 124 would face each other. If circumferential rings 124 are in phase, they may be connected by any method known in the art, such as via a connecting element. Further, one skilled in the art can appreciate that the peaks of one circumferential ring may also face a different part of an adjacent ring other than the peaks or valleys (i.e., an intermediate point). For the ease of illustration, the remaining figures do not show the circumferential rings 124 connected together via welds or connecting elements. It is understood that the circumferential rings 124 in the remaining figures are preferably connected together as described above.

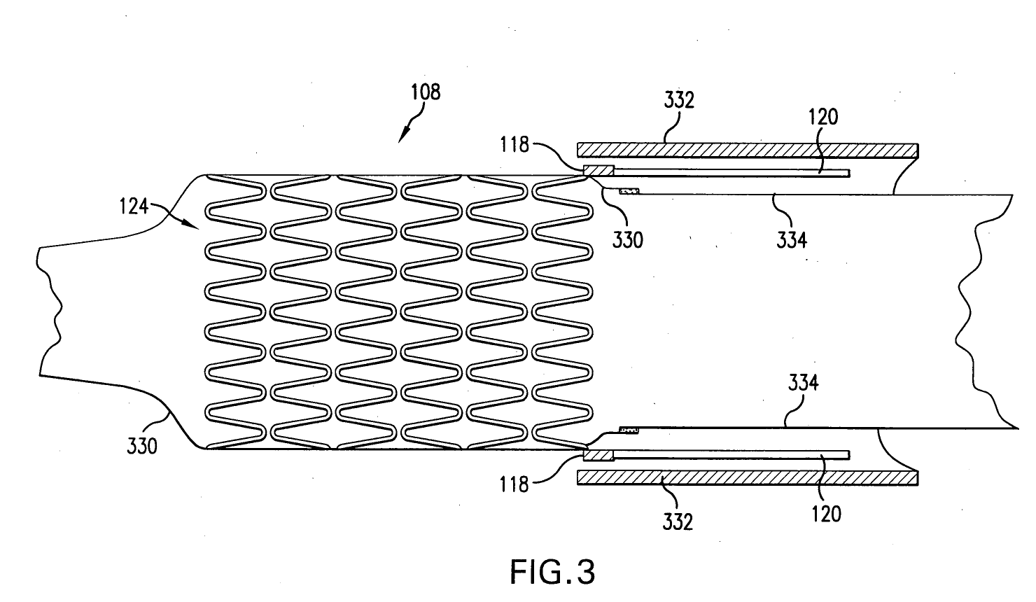

As shown in FIG. 3, for delivery to a treatment site, tubular body 108 is crimped over a balloon 330 of a balloon catheter (not shown). FIG. 3 illustrates both flaring portion 114 and tubular body 108 in an unexpanded configuration. Balloon 330 serves to expand tubular body 108, therefore allowing stent 102 to engage the branch vessel walls at the treatment site. Tubular portion 108 is accordingly balloon-expandable, while flaring portion 114 is self-expandable, as will be discussed in further detail below.

Tubular body 108 is constructed from a strong material, preferably a cobalt chrome alloy, preferably a cobalt chrome alloy with a high nickel content, such as MP35N. MP35N is a material similar to stainless steel, except that it contains more cobalt and nickel than stainless steel and does not contain iron. MP35N is preferable because it is a strong, dense, radiopaque, corrosion-resistant material. Other cobalt chrome alloys, for example, L605 and ELGILOY, may also be used. Tubular body 108 may also be constructed from stainless steel, platinum, titanium, tantalum, or any appropriate material that provides the desired strength for stent 102.

Tubular body 108 may be manufactured by any of a number of different methods known in the art. Tubular body 108 may be formed by winding a wire around a mandrel, welding or otherwise forming the stent to a desired configuration, and finally compressing the stent to an unexpanded diameter. Alternatively, tubular body 108 may be formed by laser-cutting techniques known in the art, in which a tube is cut or etched to a desired shape to form tubular body 108.

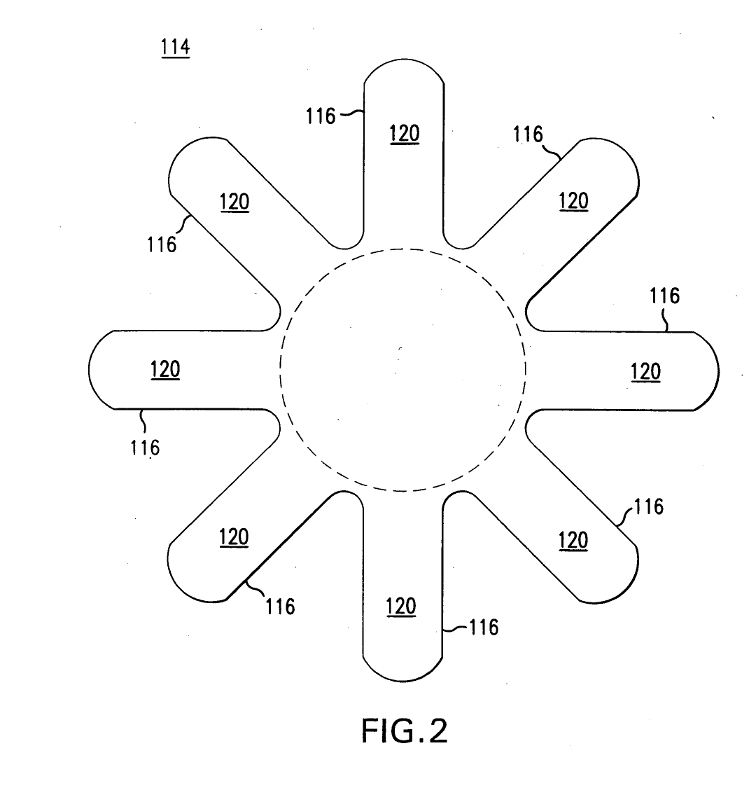

Flaring portion 114 is attached to proximal end 110 of tubular body 108. Flaring portion 114 is comprised of a plurality of flaring members 116. Flaring portion 114 contains at least one flaring member 116, but may contain any appropriate number of flaring members as needed at the particular treatment site. FIG. 2 illustrates a front view of ostium stent system 100 with flaring portion 114 in an expanded configuration, showing flaring portion 114 with eight flaring members 116. In its expanded configuration, flaring portion 114 serves to engage the wall of a great vessel at an ostium, thereby aiding in correctly positioning ostium stent system 100.

Each flaring member 116 is comprised of a short segment 118 and a long segment 120. As shown in FIG. 3 in an unexpanded configuration, short segments 118 are attached to proximal end 110 of tubular body 108 such that short segments 118 are parallel to longitudinal axis 122 of tubular body 108. Each short segment 118 has a length in the range of about 0.4 to 1.0 millimeters. Further, each long segment 120 is attached to the corresponding short segment 118 and is also parallel to longitudinal axis 122 of tubular body 108 in an unexpanded configuration. Each long segment 118 has a length in the range of about 1.0 to 5.0 millimeters. It will be recognized by those skilled in the art of stent design that the ranges provided above are exemplary only and may be varied depending on the particular application of a stent according to the present application.

Referring back to FIG. 1, which illustrates flaring portion 114 in an expanded configuration, it is apparent that short segments 118 remain parallel to longitudinal axis 122 of tubular body 108 when flaring portion 114 is deployed. However, long segments 120 become perpendicular to longitudinal axis 122 of tubular body 108 when flaring portion 114 is deployed. In their expanded configuration, long segments 120 serve to engage the wall of a great vessel at an ostium.

The advantage of having short segments 118 remain parallel to longitudinal axis 122 of tubular body 108 after deployment of flaring portion 114 is that short segments 118 thereby prevent stent 102 from lifting off balloon 330 when long segments 120 are deployed. One problem associated with stents designed for use at an ostium is that when the flaring portion is deployed, the deployment causes the body of the stent to raise off the balloon. More explicitly, when the flaring portion is deployed, the deployment exerts a radial force on the point of attachment between the flaring portion and the tubular portion. The radial force may cause the tubular portion of the stent to lift off the balloon. Subsequently, expansion of the tubular body of the stent may not be as uniform as desired since the stent was separated from the balloon. However, in the ostium stent system 100 of the present invention, short segments 118 of flaring members 116 absorb the radial force exerted by deployment of long segments 120 and therefore prevent stent 102 from lifting off balloon 330.

Another advantage of having short segments 118 remain parallel to longitudinal axis 122 of tubular body 108 after deployment of flaring portion 114 is that stresses associated with the bending of flaring portion 114 are not absorbed by the junction of flaring portion 114 and tubular body 108. As explained in more detail below, the junction between flaring portion 114 and tubular body 108 may be a welded connection. Without short segments 118, flaring portion 114 would hinge at the welded connection between tubular body 108 and flaring portion 114, causing stresses at the welded connection. Such stresses may lead to cracking in the welded connection. Such stresses are minimized with the design of the present invention.

Since flaring portion 114 is self-expandable, flaring portion 114 should be constructed from any suitable elastic material, preferably NITINOL. NITINOL is desirable because it can undergo deformations when under the influence of force, but then spring back to its original shape after the force is removed. However, any appropriate elastic material known in the art may be used such that flaring portion 114 is self-expanding. Since stent 102 contains tubular body 108 constructed from a strong material as discussed above and flaring portion 114 constructed from an elastic material, stent 102 has a tubular body of sufficient radial strength while simultaneously having a flaring portion that is flexible and self-expanding.

Referring to FIG. 3, flaring portion 114 is illustrated in an unexpanded configuration. As previously mentioned, tubular body 108 is balloon expandable, while flaring portion 114 is self expandable. The unexpanded configuration of flaring portion 114 results from a restraining structure 332 that restrains long segments 120 and holds them parallel to longitudinal axis 122 of tubular body 108. Restraining structure 332 covers only flaring portion 114, therefore covering only proximal portion 104 of stent 102. When restraining structure 332 is retracted or removed, long segments 120 are deployed and become perpendicular to longitudinal axis 122 of tubular body 108. The advantage of having restraining structure 332 cover only proximal portion 104 of stent 102 as opposed to covering the entire stent is that stent 102 has a lower profile. The lower profile allows stent 102 to properly traverse blood vessels during delivery to the treatment site. Therefore, delivery to the treatment site is smooth.

As can be seen in FIG. 3, tubular body 108 is mounted on a balloon 330 of a balloon catheter 334. In a conventional over the wire balloon catheter 334, for example, balloon 330 is bonded at its proximal end to a distal end of an outer tube and at its distal end to an inner or guide wire tube. As also shown in FIG. 3, flaring portion 114 extends over balloon catheter 334 proximal of balloon 330. Other balloon catheters, such as rapid exchange catheters, may be used as would be understood by those skilled in the art.

Stent 102 is a multiple modular prosthesis. Tubular body 108 and flaring portion 114 are multiple modules that are fixed together. Flaring portion 114 may be attached to proximal end 110 of tubular body 108 by any of a number of different methods known in the art. For example, flaring portion 114 may be welded to tubular body 108. Other methods of connecting tubular body 108 and flaring portion 114 may be used, as would be known to those of ordinary skill in the art.

Prior to welding, tubular body 108 and flaring portion 114 may be cleaned to remove the oxide layer therefrom, for example, by using an acid solution. Further, tubular body 108 and flaring portion 114 may also be electropolished to smooth out the surfaces thereof. A preferable method for welding the preferred MP35N material of tubular body 108 to the preferred NITINOL material of flaring portion 114 comprises the following steps. First, associated peaks and valleys of the respective tubular body 108 and flaring portion 114 are compressed together. Next, a pre-argon flow is run to purge the area of oxygen. This pre-argon flow is preferably run for 2-5 minutes, or such time that is sufficient to purge the area of oxygen. Next, a YAG laser is use to bond the respective peaks and valleys together. Preferably, the YAG laser is run a little towards the MP35N side (i.e., the tubular body 108 side in this example). This is preferable because it allows more of the nickel-cobalt into the weld from the MP35N, rather than the titanium from the NITINOL. It has been found that this provides a less brittle weld than if run on the NITINOL side. Next a post-weld argon flow is run to prevent oxygen from seeping into the weld while cooling. It would be understood by those of ordinary skill in the art that the welding process may be varied depending on the materials used and the performance characteristics desired, and that the above description is provided only as a presently preferred method when using MP35N for tubular body 108 and NITINOL for flaring portion 114.

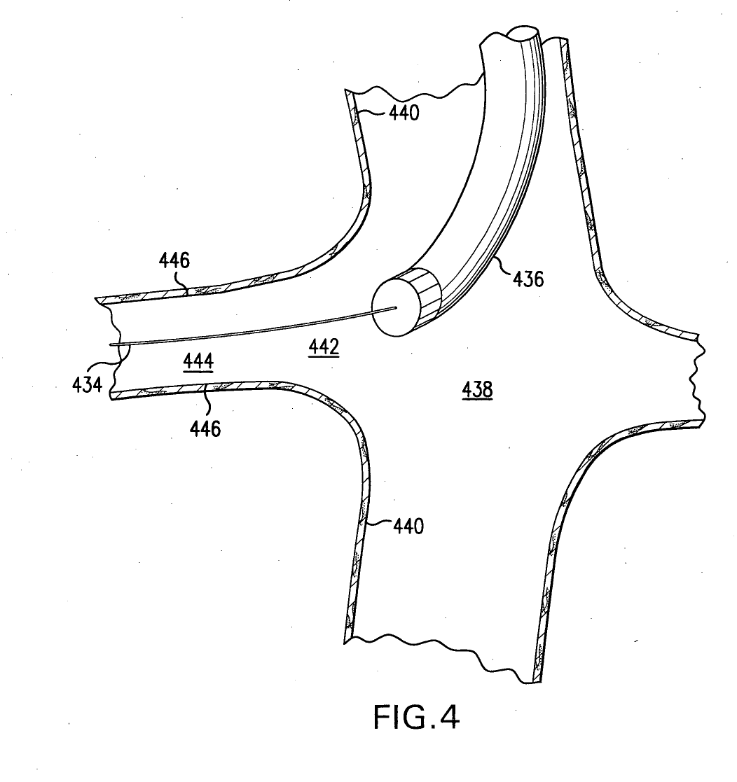

FIGS. 4-9 illustrate how ostium stent system 100 is delivered to and deployed at the treatment site. Beginning with FIG. 4, a guidewire 434 is inserted into a patient and tracked to the treatment site through a main vessel 438. A guide catheter 436 and balloon catheter 334 is then inserted over guidewire 434. Main vessel 438 is, for example, an aorta. Main vessel 438 has a wall 440, and branch vessels 444 extend from main vessel 438. An ostium 442 is formed between main vessel 438 and branch vessel 444. Applications of the present invention include ostium 442 as a sidebranch ostium or a renal ostium.

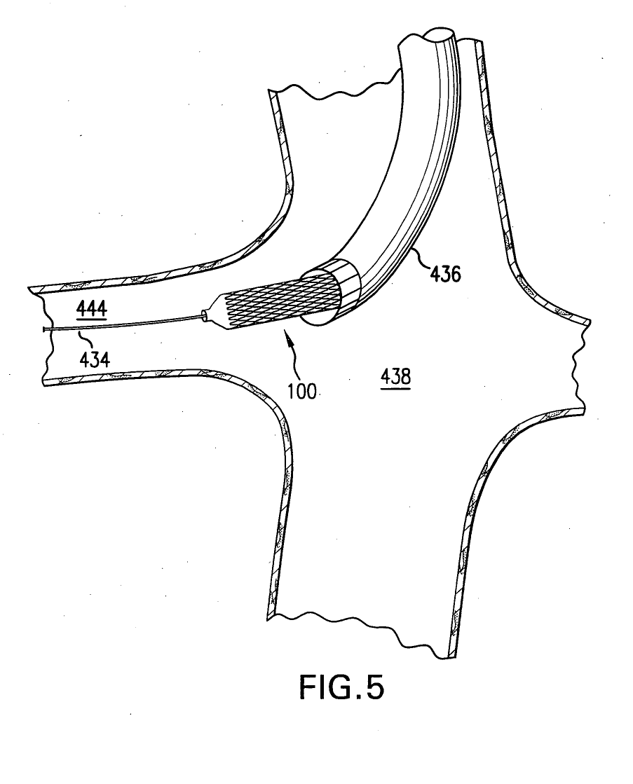

FIG. 5 shows ostium stent system 100 being tracked over guidewire 434 to the treatment site. Ostium stent system 100 is positioned just proximal to ostium 442. Radiopaque markers (not shown) may be in or attached to ostium stent system 100 to allow ostium stent system 100 to be correctly positioned.

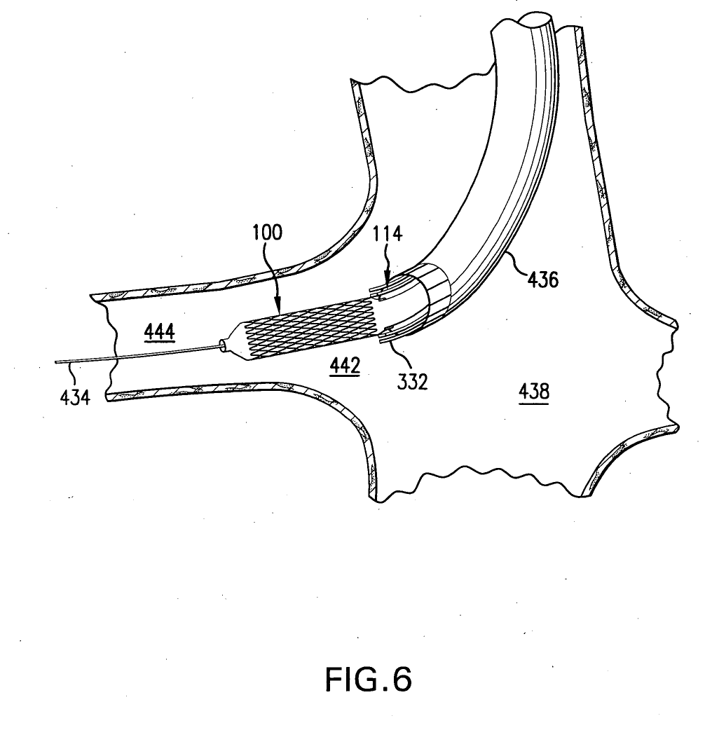

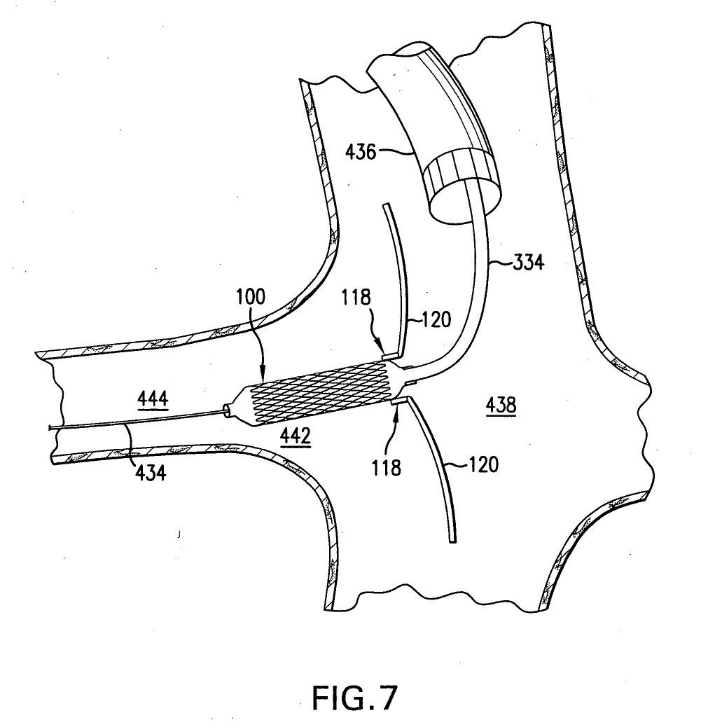

FIG. 6 shows guide catheter 436 being retracted. Ostium stent system 100 is still positioned just proximal to ostium 442. Once guide catheter 436 is pulled back, restraining structure 332 is retracted in order to deploy flaring portion 114 as shown on FIG. 7. The delivery method of restraining structure 332 is described in U.S. Pat. No. 5,824,041, the full disclosure of which is herein incorporated by reference. Essentially, restraining structure 332 is fixed to balloon catheter 334 with an external device (not shown) locked over it to hold it in place during delivery. The external device is “un-locked” to allow restraining structure 332 to be pulled back and therefore deploy flaring portion 114.

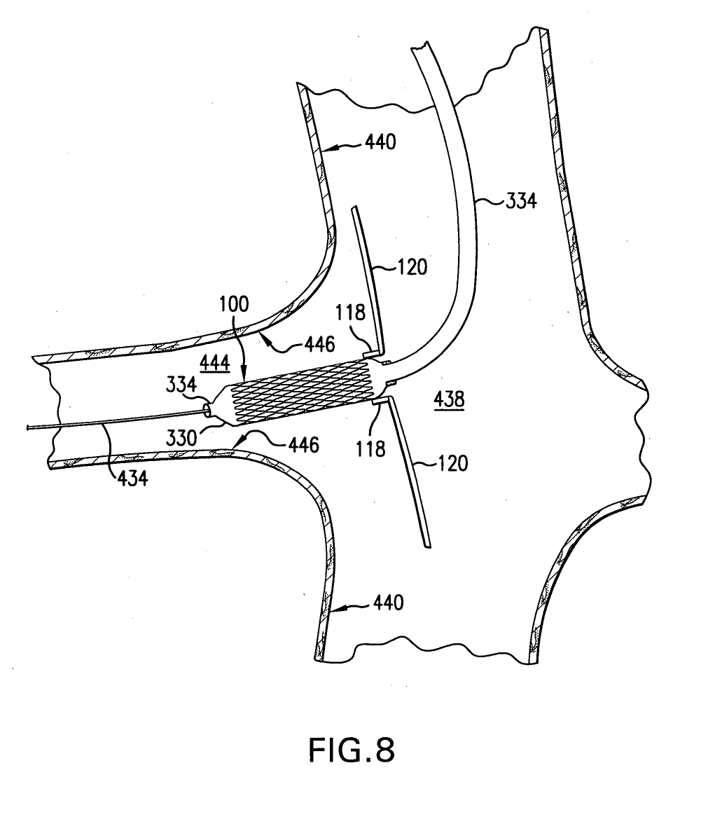

Once flaring portion 114 has been deployed, ostium stent system 100 is further advanced until long segments 120 of flaring members 116 oppose wall 440 of main vessel 438. FIG. 8 illustrates the resulting position of ostium stent system 100 such that long segments 120 of flaring members 116 oppose wall 440 of main vessel 438. Long segments 120 therefore function to self-position ostium stent system 100 since ostium stent system 100 is correctly positioned at ostium 442 once long segments 120 engage wall 440 of main vessel 438.

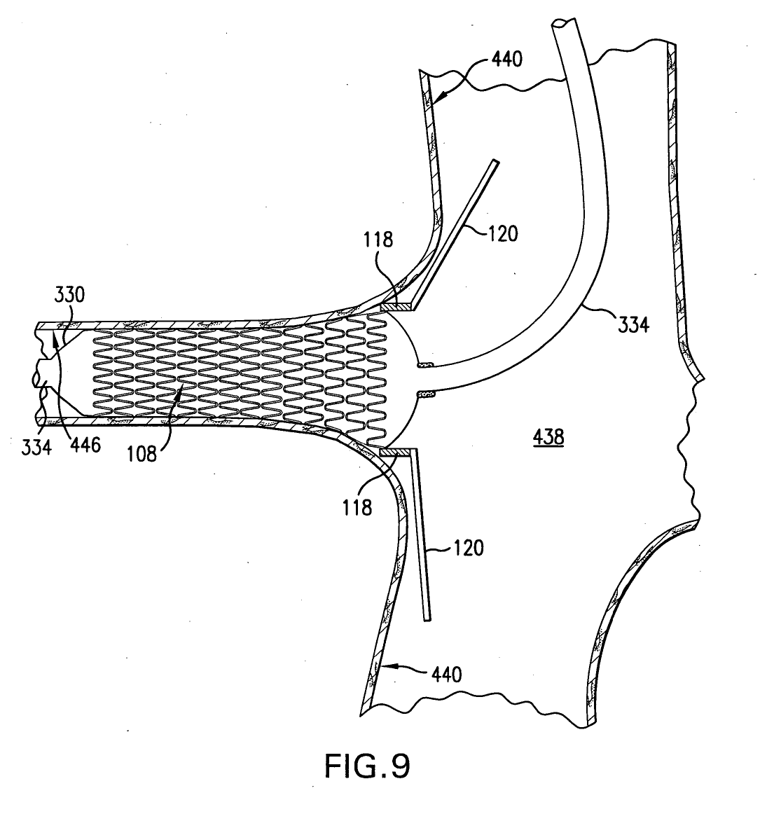

After ostium stent system 100 has been correctly positioned at ostium 442, tubular body 108 of stent 102 is expanded by inflation of balloon 330 such that stent 102 engages wall 446 of branch vessel 444. FIG. 9 illustrates tubular body 108 in its expanded position for treatment of stenosis of branch vessel 444.

While this invention has been particularly shown and described with reference to preferred embodiments thereof, it will be understood by those skilled in the art that various changes in form and details may be made therein without departing from the spirit and scope of the invention.

1. An expandable stent for use at an ostium, comprising:
a tubular body having a longitudinal axis, a proximal end and a distal end;
at least one flaring member comprised of a short segment and a long segment, wherein said at least one flaring member is attached to the proximal end of said tubular body with the short segment and the long segment both parallel to the longitudinal axis of said tubular body in an unexpanded configuration; and
wherein the short segment of said at least one flaring member remains generally parallel to the longitudinal axis of said tubular body in an expanded configuration, and the long segment of said at least one flaring member becomes generally perpendicular to the longitudinal axis of said tubular body in the expanded configuration.

2. The expandable stent of claim 1, further comprising a retaining structure covering only said at least one flaring member, wherein the removal of said retaining structure results in the expanded configuration of said at least one flaring member.

3. The expandable stent of claim 1, wherein said tubular body is constructed from a cobalt-chrome alloy

4. The expandable stent of claim 3, wherein the cobalt-chrome alloy is MP35N.

5. The expandable stent of claim 1, wherein said tubular body is constructed from stainless steel.

6. The expandable stent of claim 1, wherein said at least one flaring member is constructed from an elastic material.

7. The expandable stent of claim 1, wherein said at least one flaring member is constructed from nitinol.

8. The expandable stent of claim 1, wherein said tubular body is placed onto a balloon of a balloon catheter for expansion within a body lumen.

9. The expandable stent of claim 1, wherein the short segment of said at least one flaring member has a length in the range of about 0.4 to 1.0 millimeters.

10. The expandable stent of claim 1, wherein the long segment of said at least one flaring member has a length in the range of about 1.0 to 5.0 millimeters.

11. The expandable stent of claim 1, wherein the expandable stent is a multiple module prosthesis and the multiple modules are fixed together.

12. The expandable stent of claim 11, wherein the multiple modules are fixed together by welds.

13. An ostium stent system, comprising:
a balloon catheter, wherein the balloon catheter includes a balloon mounted on a distal portion of the balloon catheter; and
a stent mounted on the balloon, the stent including:
a tubular body having a longitudinal axis, a proximal end and a distal end, wherein the tubular body is expanded by inflation of the balloon;
at least one flaring member attached to the proximal end of said tubular body, wherein said at least one flaring member is self expandable; and
a retaining structure covering only said at least one flaring member, wherein the removal of said retaining structure results in the expanded configuration of said at least one flaring member.

14. The ostium stent system of claim 13, wherein said tubular body is constructed from a cobalt-chrome alloy.

15. The ostium stent system of claim 14, wherein the cobalt-chrome alloy is MP35N.

16. The ostium stent system of claim 13, wherein said tubular body is constructed from stainless steel.

17. The ostium stent system of claim 13, wherein said at least one flaring member is constructed from an elastic material.

18. The ostium stent system of claim 13, wherein said at least one flaring member is constructed from nitinol.

19. The ostium stent system of claim 13, wherein said at least one flaring member is comprised of a short segment and a long segment, wherein said at least one flaring member is attached to the proximal end of said tubular body with the short segment and the long segment both generally parallel to the longitudinal axis of said tubular body in an unexpanded configuration, and wherein the short segment of said at least one flaring member remains generally parallel to the longitudinal axis of said tubular body in an expanded configuration, and the long segment of said at least one flaring member becomes generally perpendicular to the longitudinal axis of said tubular body in the expanded configuration.

20. The ostium stent system of claim 19, wherein the short segment of said at least one flaring member has a length in the range of about 0.4 to 1.0 millimeters.

21. The ostium stent system of claim 19, wherein the long segment of said at least one flaring member has a length in the range of about 1.0 to 5.0 millimeters.

22. The ostium stent system of claim 14, wherein the stent is a multiple module prosthesis and the multiple modules are fixed together.

23. The ostium stent system of claim 22 wherein the multiple modules are fixed together by welds.

24. An ostium stent system, comprising:
a balloon catheter, wherein the balloon catheter includes a balloon mounted on a distal portion of the balloon catheter; and
a stent mounted on the balloon, the stent including:
a tubular body having a longitudinal axis, a proximal end and a distal end, wherein the tubular body is expanded by inflation of the balloon; and
at least one flaring member attached to the proximal end of said tubular body, wherein said at least one flaring member is self expandable.

25. The ostium stent system of claim 24 further comprising a retaining structure covering only said at least one flaring member, wherein the removal of said retaining structure results in the expanded configuration of said at least one flaring member.

26. The ostium stent system of claim 24, wherein said tubular body is constructed from a cobalt-chrome alloy

27. The ostium stent system of claim 24, wherein said tubular body is constructed from MP35N.

28. The ostium stent system of claim 24, wherein said tubular body is constructed from stainless steel.

29. The ostium stent system of claim 24, wherein said at least one flaring member is constructed from nitinol.

30. The ostium stent system of claim 24, wherein said at least one flaring member is comprised of a short segment and a long segment, wherein said at least one flaring member is attached to the proximal end of said tubular body with the short segment and the long segment both generally parallel to the longitudinal axis of said tubular body in an unexpanded configuration, and wherein the short segment of said at least one flaring member remains generally parallel to the longitudinal axis of said tubular body in an expanded configuration, and the long segment of said at least one flaring member becomes generally perpendicular to the longitudinal axis of said tubular body in the expanded configuration.

31. The ostium stent system of claim 30, wherein the short segment of said at least one flaring member has a length in the range of about 0.4 to 1.0 millimeters.

32. The ostium stent system of claim 30, wherein the long segment of said at least one flaring member has a length in the range of about 1.0 to 5.0 millimeters.

33. The ostium stent system of claim 24, wherein the stent is a multiple module prosthesis and the multiple modules are fixed together.

34. The ostium stent system of claim 33, wherein the multiple modules are fixed together by welds.
